SEQUENCE LISTING (197 sequences)

SEQ ID NO: 1 - protein, 14 aa, Artificial Sequence
  Synthetic Polypeptide
GSSTGAVTSGNYPN

SEQ ID NO: 2 - protein, 7 aa, Artificial Sequence
  Synthetic Polypeptide
GTKFLAP

SEQ ID NO: 3 - protein, 9 aa, Artificial Sequence
  Synthetic Polypeptide
VLWYSNRWV

SEQ ID NO: 4 - protein, 5 aa, Artificial Sequence
  Synthetic Polypeptide
KYAMN

SEQ ID NO: 5 - protein, 19 aa, Artificial Sequence
  Synthetic Polypeptide
RIRSKYNNYATYYADSVKD

SEQ ID NO: 6 - protein, 14 aa, Artificial Sequence
  Synthetic Polypeptide
HGNFGNSYISYWAY

SEQ ID NO: 7 - protein, 125 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 8 - protein, 109 aa, Artificial Sequence
  Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 9 - protein, 249 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 10 - protein, 17 aa, Artificial Sequence
  Synthetic Polypeptide
KSSQSVLDSSTNKNSLA

SEQ ID NO: 11 - protein, 7 aa, Artificial Sequence
  Synthetic Polypeptide
WASTRES

SEQ ID NO: 12 - protein, 9 aa, Artificial Sequence
  Synthetic Polypeptide
QQSAHFPIT

SEQ ID NO: 13 - protein, 5 aa, Artificial Sequence
  Synthetic Polypeptide
NYGMN

SEQ ID NO: 14 - protein, 17 aa, Artificial Sequence
  Synthetic Polypeptide
WINTYTGEPTYADKFQG

SEQ ID NO: 15 - protein, 13 aa, Artificial Sequence
  Synthetic Polypeptide
WSWSDGYYVYFDY

SEQ ID NO: 16 - protein, 122 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 17 - protein, 122 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 18 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 19 - protein, 113 aa, Artificial Sequence
  Synthetic Polypeptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 20 - protein, 505 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 530 aa, Artificial Sequence
  Synthetic Polypeptide
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 22 - protein, 993 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 511 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 24 - protein, 16 aa, Artificial Sequence
  Synthetic Polypeptide
RSSQSLVHSDGNTYLS

SEQ ID NO: 25 - protein, 7 aa, Artificial Sequence
  Synthetic Polypeptide
RISRRFS

SEQ ID NO: 26 - protein, 9 aa, Artificial Sequence
  Synthetic Polypeptide
MQSTHVPRT

SEQ ID NO: 27 - protein, 5 aa, Artificial Sequence
  Synthetic Polypeptide
NYGMH

SEQ ID NO: 28 - protein, 17 aa, Artificial Sequence
  Synthetic Polypeptide
VIWYDGSDKYYADSVRG

SEQ ID NO: 29 - protein, 15 aa, Artificial Sequence
  Synthetic Polypeptide
DGYDILTGNPRDFDY

SEQ ID NO: 30 - protein, 124 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 31 - protein, 112 aa, Artificial Sequence
  Synthetic Polypeptide
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 32 - protein, 251 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 33 - protein, 506 aa, Artificial Sequence
  Synthetic Polypeptide
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 34 - protein, 5 aa, Artificial Sequence
  Synthetic Polypeptide
DYYMT

SEQ ID NO: 35 - protein, 17 aa, Artificial Sequence
  Synthetic Polypeptide
YISSSGSTIYYADSVKG

SEQ ID NO: 36 - protein, 8 aa, Artificial Sequence
  Synthetic Polypeptide
DRNSHFDY

SEQ ID NO: 37 - protein, 11 aa, Artificial Sequence
  Synthetic Polypeptide
RASQGINTWLA

SEQ ID NO: 38 - protein, 7 aa, Artificial Sequence
  Synthetic Polypeptide
GASGLQS

SEQ ID NO: 39 - protein, 9 aa, Artificial Sequence
  Synthetic Polypeptide
QQAKSFPRT

SEQ ID NO: 40 - protein, 117 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 41 - protein, 107 aa, Artificial Sequence
  Synthetic Polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 42 - protein, 239 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 43 - protein, 494 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 982 aa, Artificial Sequence
  Synthetic Polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 982 aa, Artificial Sequence; identical to SEQ ID NO: 44
  Synthetic Polypeptide

SEQ ID NO: 46 - protein, 5 aa, Artificial Sequence
  Synthetic Polypeptide
SYGMH

SEQ ID NO: 47 - protein, 17 aa, Artificial Sequence
  Synthetic Polypeptide
FIWYEGSNKYYAESVKD

SEQ ID NO: 48 - protein, 16 aa, Artificial Sequence
  Synthetic Polypeptide
RAGIIGTIGYYYGMDV

SEQ ID NO: 49 - protein, 11 aa, Artificial Sequence
  Synthetic Polypeptide
SGDRLGEKYTS

SEQ ID NO: 50 - protein, 7 aa, Artificial Sequence
  Synthetic Polypeptide
QDTKRPS

SEQ ID NO: 51 - protein, 9 aa, Artificial Sequence
  Synthetic Polypeptide
QAWESSTVV

SEQ ID NO: 52 - protein, 125 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 53 - protein, 107 aa, Artificial Sequence
  Synthetic Polypeptide
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 54 - protein, 247 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 55 - protein, 507 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 56 - protein, 7 aa, Artificial Sequence
  Synthetic Polypeptide
NARMGVS

SEQ ID NO: 57 - protein, 16 aa, Artificial Sequence
  Synthetic Polypeptide
HIFSNDEKSYSTSLKN

SEQ ID NO: 58 - protein, 14 aa, Artificial Sequence
  Synthetic Polypeptide
IVGYGSGWYGFFDY

SEQ ID NO: 59 - protein, 11 aa, Artificial Sequence
  Synthetic Polypeptide
RASQGIRNDLG

SEQ ID NO: 60 - protein, 7 aa, Artificial Sequence
  Synthetic Polypeptide
AASTLQS

SEQ ID NO: 61 - protein, 9 aa, Artificial Sequence
  Synthetic Polypeptide
LQHNSYPLT

SEQ ID NO: 62 - protein, 124 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 63 - protein, 107 aa, Artificial Sequence
  Synthetic Polypeptide
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 64 - protein, 246 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 65 - protein, 501 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 66 - protein, 989 aa, Artificial Sequence
  Synthetic Polypeptide
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 5 aa, Artificial Sequence
  Synthetic Polypeptide
SYYWS

SEQ ID NO: 68 - protein, 16 aa, Artificial Sequence
  Synthetic Polypeptide
YVYYSGTTNYNPSLKS

SEQ ID NO: 69 - protein, 10 aa, Artificial Sequence
  Synthetic Polypeptide
IAVTGFYFDY

SEQ ID NO: 70 - protein, 12 aa, Artificial Sequence
  Synthetic Polypeptide
RASQRVNNNYLA

SEQ ID NO: 71 - protein, 7 aa, Artificial Sequence
  Synthetic Polypeptide
GASSRAT

SEQ ID NO: 72 - protein, 9 aa, Artificial Sequence
  Synthetic Polypeptide
QQYDRSPLT

SEQ ID NO: 73 - protein, 118 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 74 - protein, 108 aa, Artificial Sequence
  Synthetic Polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 75 - protein, 241 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 76 - protein, 496 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 77 - protein, 982 aa, Artificial Sequence
  Synthetic Polypeptide
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 5 aa, Artificial Sequence
  Synthetic Polypeptide
SYGMH

SEQ ID NO: 79 - protein, 17 aa, Artificial Sequence
  Synthetic Polypeptide
VISYEGSNKYYAESVKG

SEQ ID NO: 80 - protein, 13 aa, Artificial Sequence
  Synthetic Polypeptide
DRGTIFGNYGLEV

SEQ ID NO: 81 - protein, 16 aa, Artificial Sequence
  Synthetic Polypeptide
RSSQSLLHKNAFNYLD

SEQ ID NO: 82 - protein, 7 aa, Artificial Sequence
  Synthetic Polypeptide
LGSNRAS

SEQ ID NO: 83 - protein, 9 aa, Artificial Sequence
  Synthetic Polypeptide
MQALQTPFT

SEQ ID NO: 84 - protein, 122 aa, Artificial Sequence
  Synthetic Polypeptide
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 85 - protein, 112 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 86 - protein, 503 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 87 - protein, 991 aa, Artificial Sequence
  Synthetic Polypeptide
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 5 aa, Artificial Sequence
  Synthetic Polypeptide
NHIIH

SEQ ID NO: 89 - protein, 17 aa, Artificial Sequence
  Synthetic Polypeptide
YINPYPGYHAYNEKFQG

SEQ ID NO: 90 - protein, 12 aa, Artificial Sequence
  Synthetic Polypeptide
DGYYRDTDVLDY

SEQ ID NO: 91 - protein, 11 aa, Artificial Sequence
  Synthetic Polypeptide
QASQDISNYLN

SEQ ID NO: 92 - protein, 7 aa, Artificial Sequence
  Synthetic Polypeptide
YTSRLHT

SEQ ID NO: 93 - protein, 9 aa, Artificial Sequence
  Synthetic Polypeptide
QQGNTLPWT

SEQ ID NO: 94 - protein, 121 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 95 - protein, 107 aa, Artificial Sequence
  Synthetic Polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 96 - protein, 243 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 97 - protein, 986 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 504 aa, Artificial Sequence
  Synthetic Polypeptide
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 99 - protein, 986 aa, Artificial Sequence; identical to SEQ ID NO: 97
  Synthetic Polypeptide

SEQ ID NO: 100 - protein, 5 aa, Artificial Sequence
  Synthetic Polypeptide
DYYMY

SEQ ID NO: 101 - protein, 17 aa, Artificial Sequence
  Synthetic Polypeptide
IISDAGYYTYYSDIIKG

SEQ ID NO: 102 - protein, 12 aa, Artificial Sequence
  Synthetic Polypeptide
GFPLLRHGAMDY

SEQ ID NO: 103 - protein, 11 aa, Artificial Sequence
  Synthetic Polypeptide
KASQNVDANVA

SEQ ID NO: 104 - protein, 7 aa, Artificial Sequence
  Synthetic Polypeptide
SASYVYW

SEQ ID NO: 105 - protein, 9 aa, Artificial Sequence
  Synthetic Polypeptide
QQYDQQLIT

SEQ ID NO: 106 - protein, 121 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 107 - protein, 107 aa, Artificial Sequence
  Synthetic Polypeptide
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 108 - protein, 243 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 109 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 986 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 984 aa, Artificial Sequence
  Synthetic Polypeptide
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 5 aa, Artificial Sequence
  Synthetic Polypeptide
DYYMY

SEQ ID NO: 113 - protein, 17 aa, Artificial Sequence
  Synthetic Polypeptide
IISDGGYYTYYSDIIKG

SEQ ID NO: 114 - protein, 12 aa, Artificial Sequence
  Synthetic Polypeptide
GFPLLRHGAMDY

SEQ ID NO: 115 - protein, 11 aa, Artificial Sequence
  Synthetic Polypeptide
KASQNVDTNVA

SEQ ID NO: 116 - protein, 7 aa, Artificial Sequence
  Synthetic Polypeptide
SASYVYW

SEQ ID NO: 117 - protein, 9 aa, Artificial Sequence
  Synthetic Polypeptide
QQYDQQLIT

SEQ ID NO: 118 - protein, 121 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 119 - protein, 107 aa, Artificial Sequence
  Synthetic Polypeptide
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 120 - protein, 243 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 121 - protein, 498 aa, Artificial Sequence
  Synthetic Polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 122 - protein, 986 aa, Artificial Sequence
  Synthetic Polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 5 aa, Artificial Sequence
  Synthetic Polypeptide
TYAMS

SEQ ID NO: 124 - protein, 17 aa, Artificial Sequence
  Synthetic Polypeptide
AISGSGGRTFYAESVEG

SEQ ID NO: 125 - protein, 11 aa, Artificial Sequence
  Synthetic Polypeptide
HDYSNYPYFDY

SEQ ID NO: 126 - protein, 12 aa, Artificial Sequence
  Synthetic Polypeptide
RASQSVRSTYLA

SEQ ID NO: 127 - protein, 7 aa, Artificial Sequence
  Synthetic Polypeptide
GASSRAT

SEQ ID NO: 128 - protein, 9 aa, Artificial Sequence
  Synthetic Polypeptide
QQYGDLPFT

SEQ ID NO: 129 - protein, 120 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 130 - protein, 108 aa, Artificial Sequence
  Synthetic Polypeptide
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 131 - protein, 986 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 5 aa, Artificial Sequence
  Synthetic Polypeptide
GYYMH

SEQ ID NO: 133 - protein, 17 aa, Artificial Sequence
  Synthetic Polypeptide
WINPNSGGTKYAQKFQG

SEQ ID NO: 134 - protein, 16 aa, Artificial Sequence
  Synthetic Polypeptide
DRITVAGTYYYYGMDV

SEQ ID NO: 135 - protein, 11 aa, Artificial Sequence
  Synthetic Polypeptide
RASQGVNNWLA

SEQ ID NO: 136 - protein, 7 aa, Artificial Sequence
  Synthetic Polypeptide
TASSLQS

SEQ ID NO: 137 - protein, 9 aa, Artificial Sequence
  Synthetic Polypeptide
QQANSFPIT

SEQ ID NO: 138 - protein, 125 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 139 - protein, 107 aa, Artificial Sequence
  Synthetic Polypeptide
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 140 - protein, 247 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 141 - protein, 502 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 142 - protein, 990 aa, Artificial Sequence
  Synthetic Polypeptide
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 125 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 144 - protein, 107 aa, Artificial Sequence; identical to SEQ ID NO: 139
  Synthetic Polypeptide

SEQ ID NO: 145 - protein, 247 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 146 - protein, 502 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 990 aa, Artificial Sequence
  Synthetic Polypeptide
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 5 aa, Artificial Sequence
  Synthetic Polypeptide
NHGMH

SEQ ID NO: 149 - protein, 17 aa, Artificial Sequence
  Synthetic Polypeptide
GIWSEGSNKYYADAVKG

SEQ ID NO: 150 - protein, 12 aa, Artificial Sequence
  Synthetic Polypeptide
ATYTTGWSYFDY

SEQ ID NO: 151 - protein, 11 aa, Artificial Sequence
  Synthetic Polypeptide
SGDKLGDKYAS

SEQ ID NO: 152 - protein, 7 aa, Artificial Sequence
  Synthetic Polypeptide
QDAKRPS

SEQ ID NO: 153 - protein, 9 aa, Artificial Sequence
  Synthetic Polypeptide
QAFHQSTWV

SEQ ID NO: 154 - protein, 121 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSS

SEQ ID NO: 155 - protein, 106 aa, Artificial Sequence
  Synthetic Polypeptide
SYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVL

SEQ ID NO: 156 - protein, 985 aa, Artificial Sequence
  Synthetic Polypeptide
QVQLVESGGGVVQPGRSLRLSCAASGFTFSNHGMHWVRQAPGKCLEWVAGIWSEGSNKYYADAVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDAKRPSGIPERFSGSNSGNTATLTISGTQAMDEADYYCQAFHQSTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 5 aa, Artificial Sequence
  Synthetic Polypeptide
NHAMH

SEQ ID NO: 158 - protein, 17 aa, Artificial Sequence
  Synthetic Polypeptide
GIWSEGSNKYYAESVKG

SEQ ID NO: 159 - protein, 12 aa, Artificial Sequence
  Synthetic Polypeptide
ATYTTGWSYFDY

SEQ ID NO: 160 - protein, 11 aa, Artificial Sequence
  Synthetic Polypeptide
SGDKLGDKYAS

SEQ ID NO: 161 - protein, 7 aa, Artificial Sequence
  Synthetic Polypeptide
QDRKRPS

SEQ ID NO: 162 - protein, 9 aa, Artificial Sequence
  Synthetic Polypeptide
QAYDASTWV

SEQ ID NO: 163 - protein, 121 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSS

SEQ ID NO: 164 - protein, 106 aa, Artificial Sequence
  Synthetic Polypeptide
SYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVL

SEQ ID NO: 165 - protein, 985 aa, Artificial Sequence
  Synthetic Polypeptide
QVQLVESGGGVVQPGRSLRLSCAASGFTFSNHAMHWVRQAPGKCLEWVAGIWSEGSNKYYAESVKGRFTISRDNSKNTLYLQMNSLRAEDTAVYYCARATYTTGWSYFDYWGQGTLVTVSSGGGGSGGGGSGGGGSSYELTQPPSVSVSPGQTASITCSGDKLGDKYASWYQQKSGQSPVLVIYQDRKRPSGIPERFSGSNSGNTATLTISGTQAMDEADYYCQAYDASTWVFGCGTQLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 5 aa, Artificial Sequence
  Synthetic Polypeptide
GYYWS

SEQ ID NO: 167 - protein, 16 aa, Artificial Sequence
  Synthetic Polypeptide
DIDASGSTKYNPSLKS

SEQ ID NO: 168 - protein, 12 aa, Artificial Sequence
  Synthetic Polypeptide
KKYSTVWSYFDN

SEQ ID NO: 169 - protein, 11 aa, Artificial Sequence
  Synthetic Polypeptide
SGDKLGDKYAS

SEQ ID NO: 170 - protein, 7 aa, Artificial Sequence
  Synthetic Polypeptide
QDRKRPS

SEQ ID NO: 171 - protein, 9 aa, Artificial Sequence
  Synthetic Polypeptide
QAWGSSTAV

SEQ ID NO: 172 - protein, 120 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 173 - protein, 106 aa, Artificial Sequence
  Synthetic Polypeptide
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 174 - protein, 984 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 5 aa, Artificial Sequence
  Synthetic Polypeptide
GYYWS

SEQ ID NO: 176 - protein, 16 aa, Artificial Sequence
  Synthetic Polypeptide
DIDYSGSTKYNPSLKS

SEQ ID NO: 177 - protein, 12 aa, Artificial Sequence
  Synthetic Polypeptide
KKYSTVWSYFDY

SEQ ID NO: 178 - protein, 11 aa, Artificial Sequence
  Synthetic Polypeptide
SGDKLGDKYAN

SEQ ID NO: 179 - protein, 7 aa, Artificial Sequence
  Synthetic Polypeptide
HDNKRPS

SEQ ID NO: 180 - protein, 9 aa, Artificial Sequence
  Synthetic Polypeptide
QAYGISSAV

SEQ ID NO: 181 - protein, 120 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSS

SEQ ID NO: 182 - protein, 106 aa, Artificial Sequence
  Synthetic Polypeptide
SYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVL

SEQ ID NO: 183 - protein, 984 aa, Artificial Sequence
  Synthetic Polypeptide
QVQLQQWGAGLLKPSETLSLTCAVYGGSFSGYYWSWIRQPPGKCLEWIGDIDYSGSTKYNPSLKSRVTISLDTSKNQFSLKLNSVTAADTAVYFCARKKYSTVWSYFDYWGQGTLVTVSSGGGGSGGGGSGGGGSSYELTQPASASVSPGQTASITCSGDKLGDKYANWYQQKPGQSPILVIYHDNKRPSGIPERFSGSNSGNTATLTISGTQAMDEADYYCQAYGISSAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 989 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 987 aa, Artificial Sequence
  Synthetic Polypeptide
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 501 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 187 - protein, 989 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 987 aa, Artificial Sequence
  Synthetic Polypeptide
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 250 aa, Artificial Sequence
  Synthetic Polypeptide
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 190 - protein, 250 aa, Artificial Sequence
  Synthetic Polypeptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 191 - protein, 244 aa, Artificial Sequence
  Synthetic Polypeptide
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKS

SEQ ID NO: 192 - protein, 250 aa, Artificial Sequence
  Synthetic Polypeptide
SEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 193 - protein, 484 aa, Artificial Sequence
  Synthetic Polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 242 aa, Artificial Sequence
  Synthetic Polypeptide
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLS

SEQ ID NO: 195 - protein, 250 aa, Artificial Seuqence; identical to SEQ ID NO: 192
  Synthetic Polypeptide

SEQ ID NO: 196 - protein, 484 aa, Artificial Sequence; identical to SEQ ID NO: 193
  Synthetic Polypeptide

SEQ ID NO: 197 - protein, 4 aa, Artificial Sequence
  Synthetic Linker
GGGG

(19) United States
(12) Patent Application Publication
(10) Pub. No.: US 2023/0348596 A1
(43) Pub. Date: Nov. 2, 2023

(54) PHARMACEUTICAL FORMULATION

(71) Applicant: AMGEN INC., Thousand Oaks, CA (US)

(72) Inventor: Bharadwaj Jagannathan, Thousand Oaks, CA (US)

(21) Appl. No.: 18/022,685

(22) PCT Filed: Aug. 23, 2021
(86) PCT No.: PCT/US21/47181

§ 371 Date: Feb. 22, 2023

Related U.S. Application Data
(60) Provisional application No. 63/197,020, filed on Jun. 4, 2021.
(60) Provisional application No. 63/069,432, filed on Aug. 24, 2020.

(51) Int. Cl. C07K 16/28; A61K 47/20; A61K 47/26; A61K 47/18; A61K 9/19; C07K 16/30

(52) U.S. Cl. CPC C07K 16/2809; A61K 47/20; A61K 47/26; A61K 47/183; A61K 9/19; C07K 16/3069; C07K 16/28; C07K 16/2803; C07K 16/2851; C07K 16/2878; C07K 16/30; C07K 2317/31; C07K 2317/94

(57) ABSTRACT

The disclosure provides a formulation comprising, e.g., a bispecific antibody construct, a buffer, a saccharide, a surfactant, and methionine.

### CROSS REFERENCE TO RELATED APPLICATION AND INCORPORATION BY REFERENCE OF MATERIAL SUBMITTED ELECTRONICALLY

This application claims priority to U.S. Provisional Patent Application Nos. 63/069,432 and 63/197,020, filed Aug. 24, 2020, and Jun. 4, 2021, respectively, which are hereby incorporated by reference in their entirety.

Incorporated by reference in its entirety is a computer-readable nucleotide/amino acid sequence listing submitted concurrently herewith and identified as follows: ASCII (text) file named “55632A_Seqlisting.txt”, 362,796 bytes created Aug. 11, 2021.

### FIELD OF THE INVENTION

The present disclosure is in the field of stable bispecific antibody construct formulations.

### BACKGROUND

Protein-based pharmaceuticals are among the fastest growing therapeutic agents in (pre)clinical development and as commercial products. In comparison with small chemical drugs, protein pharmaceuticals have high specificity and activity at relatively low concentrations, and typically provide for therapy of high impact diseases such as various cancers, auto-immune diseases, and metabolic disorders (Roberts, Trends Biotechnol. 2014 July; 32(7):372-80, Wang, Int J Pharm. 1999 Aug. 20; 185(2):129-88).

Protein-based pharmaceuticals, such as recombinant proteins, can now be obtained in high purity when first manufactured due to advances in commercial scale purification processes. However, proteins are only marginally stable and are highly susceptible to degradation, both chemical and physical. Chemical degradation refers to modifications involving covalent bonds, such as deamidation, oxidation, cleavage, clipping/fragmentation, formation of new disulfide bridges, hydrolysis, isomerization, or deglycosylation. Physical degradation includes protein unfolding, undesirable adsorption to surfaces, and aggregation. Dealing with these physical and chemical instabilities is one of the most challenging tasks in the development of protein pharmaceuticals (Chi et al., Pharm Res, Vol. 20, No. 9, September 2003, pp. 1325-1336, Roberts, Trends Biotechnol. 2014 July; 32(7):372-80).

There is a need in the art for pharmaceutical formulations that provide for enhanced stabilization of therapeutic proteins during shipping and storage.

### SUMMARY

Protein-based pharmaceuticals including bispecific and multispecific antibody constructs that bind to two (or more) different antigens simultaneously, such as bispecific T cell engaging (BITE®) antibody constructs, are prone to protein instability. This extends to those antibody constructs comprising half-life extending formats (HLE formats) which include the single chain Fc format (designated scFc), the hetero Fc (also designated as hetFc or heterodimeric Fc, hFc) format, and the fusion of human serum albumin (also designated as HSA or hALB). Bispecific antibody constructs, including BiTE HLE constructs, are susceptible to aggregation (i.e., the formation of high molecular weight (HMW) species) when frozen and stored at, e.g., at −30° C. This instability necessitates storage at −70° C. to minimize aggregation. The requirement to maintain a temperature of −70° C., however, raises significant storage and transportation challenges, as special equipment and procedures are necessary to consistently maintain the low temperature.

The present disclosure is based, at least in part, on the surprising discovery that methionine reduces the formation of bispecific antibody construct HMW species when frozen and stored at −30° C. As shown in the Example described herein, methionine reduced aggregation (i.e., the appearance of HMW species) by about 25% to about 85% in frozen formulations comprising bispecific antibody constructs stored at −30° C.; a similar protective effect was not detected in liquid formulations stored at 4° C. or 40° C. for a similar time frame. The materials and methods described herein provide a significant technical advantage by, e.g., simplifying the equipment and procedures required to store and transport bispecific antibody constructs while minimizing aggregation.

In various aspects, the disclosure provides a pharmaceutical formulation comprising a bispecific antibody construct, a saccharide, a surfactant, a buffer, and methionine present at a molar ratio of methionine to bispecific antibody construct of about 10× to about 5000× (e.g., a molar ratio of methionine to bispecific antibody construct of about 50× to about 5000×). Optionally, the formulation may comprise about 10 mM to about 200 mM methionine. The pH of the formulation is from about 4 to about 7 (e.g., about 4 to about 6, such as about 4.2). Optionally, the saccharide is sucrose, the surfactant is polysorbate 80, and/or the buffer is a glutamate buffer. In various aspects, the bispecific antibody construct is present in the formulation at a concentration of from about 1 mg/ml to about 20 mg/ml. In various embodiments, the formulation is frozen. For example, the disclosure provides a frozen pharmaceutical formulation comprising about 1 mg/mL to about 20 mg/mL bispecific antibody construct, sucrose, glutamic acid, polysorbate 80, and about 10 mM to about 200 mM methionine, wherein the pH of the formulation is from about 4 to about 7 (e.g., about 4 to about 6). In alternative embodiments, the formulation is a thawed formulation or is lyophilized. In various aspects, the bispecific antibody construct comprises the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

The disclosure further provides a method of treating cancer in a subject in need thereof comprising administering a formulation of the disclosure to the subject. The use of the formulation in any of the methods disclosed herein or for the preparation of medicaments for administration according to any of the methods disclosed herein is specifically contemplated. The disclosure also provides a formulation as described herein for use in treating cancer.

Additionally, the disclosure provides a method comprising (a) preparing a formulation comprising a bispecific antibody construct, methionine, and a buffer, wherein the methionine present at a molar ratio of methionine to bispecific antibody construct of about 10× to about 5000×; (b) freezing the formulation of (a); and (c) storing the formulation of (b) at a temperature of about −10° C. to about −40° C. In various aspects of the disclosure, steps (b) and (c) are performed at a temperature of about −20° C. to about −35° C. (e.g., 30° C.) and/or step (c) comprises storing the formulation for at least one month. Optionally, the method further comprises (d) thawing the formulation of (c); and (e) lyophilizing the formulation of (d). In some embodiments, the formulation comprises methionine at molar ratio of methionine to bispecific antibody construct of about 50× to about 5000×, such as in an amount of about 10 mM to about 200 mM methionine, and/or the bispecific antibody construct is present at a concentration of from about 1 mg/ml to about 20 mg/ml.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. For example, “a bispecific antibody construct” is understood to represent one or more bispecific antibody constructs. As such, the terms “a” (or “an”), “one or more,” and “at least one” can be used interchangeably herein. The term “or” should be understood to encompass items in the alternative or together, unless context unambiguously requires otherwise.

It should also be understood that when describing a range of values, the disclosure contemplates individual values found within the range. For example, “a pH from about pH 4 to about pH 6,” could be, but is not limited to, pH 4.2, 4.6, 5.2, 5.5, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE FIGURES

FIG. 1 is a graph showing the increase in percent (%) high molecular weight (HMW) species of BiTE®-1, BiTE®-2, BiTE®-3, BiTE®-4, BiTE®-5, BiTE®-6, BiTE®-7, and BiTE®-8 in a formulation comprising 10 mM glutamate, 9% sucrose, 0.01% polysorbate 80 (PS80) (pH 4.2), with (gray bar on left) and without (black bar on right) 50 mM methionine, after one month storage at −15° C. Storage at −15° C. represents accelerated stress conditions for −30° C. storage. HMW species were detected using SE-UHPLC.

FIG. 2 is a graph showing the increase in percent (%) high molecular weight (HMW) species of BiTE®-1, BiTE®-2, BiTE®-3, BiTE®-4, BiTE®-5, BiTE®-6, BiTE®-7, and BiTE®-8 in a formulation comprising 10 mM glutamate, 9% sucrose, 0.01% polysorbate 80 (PS80) (pH 4.2), with and without 50 mM methionine, after one month liquid storage at 4° C. HMW species were detected using SE-UHPLC. Measurements illustrated in the graph for each bispecific antibody construct include (from left to right): % HMW detected at time 0 in the formulation without methionine, % HMW detected at four weeks in the formulation without methionine, % HMW detected at time 0 in the formulation with methionine, and % HMW detected at four weeks in the formulation with methionine.

FIG. 3 is a graph showing the increase in percent (%) high molecular weight (HMW) species of BiTE®-1, BiTE®-2, BiTE®-3, BiTE®-4, BiTE®-5, BiTE®-6, BiTE®-7, and BiTE®-8 in a formulation comprising 10 mM glutamate, 9% sucrose, 0.01% polysorbate 80 (PS80) (pH 4.2), with and without 50 mM methionine, after one month liquid storage at 40° C. HMW species were detected using SE-UHPLC. Measurements illustrated in the graph for each bispecific antibody construct include (from left to right): % HMW detected at time 0 in the formulation without methionine, % HMW detected at four weeks in the formulation without methionine, % HMW detected at time 0 in the formulation with methionine, and % HMW detected at four weeks in the formulation with methionine.

FIG. 4 is a graph showing the increase in percent (%) high molecular weight (HMW) species of BiTE®-1, BiTE®-2, BiTE®-3, BiTE®-4, BiTE®-5, BiTE®-6, BiTE®-7, and BiTE®-8 in a formulation comprising 10 mM glutamate, 9% sucrose, 0.01% polysorbate 80 (PS80) (pH 4.2), with and without 50 mM methionine, after one month storage in lyophilized form at 4° C. HMW species were detected using SE-UHPLC. Measurements illustrated in the graph for each bispecific antibody construct include (from left to right): % HMW detected at time 0 in the formulation without methionine, % HMW detected at four weeks in the formulation without methionine, % HMW detected at time 0 in the formulation with methionine, and % HMW detected at four weeks in the formulation with methionine.

FIG. 5 is a graph showing the increase in percent (%) high molecular weight (HMW) species of BiTE®-1, BiTE®-2, BiTE®-3, BiTE®-4, BiTE®-5, BiTE®-6, BiTE®-7, and BiTE®-8 in a formulation comprising 10 mM glutamate, 9% sucrose, 0.01% polysorbate 80 (PS80) (pH 4.2), with and without 50 mM methionine, after one month storage in lyophilized form at 40° C. HMW species were detected using SE-UHPLC. Measurements illustrated in the graph for each bispecific antibody construct include (from left to right): % HMW detected at time 0 in the formulation without methionine, % HMW detected at four weeks in the formulation without methionine, % HMW detected at time 0 in the formulation with methionine, and % HMW detected at four weeks in the formulation with methionine.

FIG. 6A is a graph showing increase in aggregation levels of various antibody constructs (BiTE®s) after one-month frozen storage at −20° C. in a formulation comprising 10 mM glutamate, 9% sucrose, 0.01% polysorbate 80 (PS80) (pH 4.2), with and without amino acid excipients (10 mM concentration). The bars for each BiTE® represent, from left to right, no amino acid excipient (control), arginine, histidine, lysine, and proline.

FIG. 6B is a graph showing the increase in BiTE®-5 aggregation levels after one-month frozen storage at −20° C. in a formulation comprising 10 mM glutamate, 9% sucrose, 0.01% polysorbate 80 (PS80) (pH 4.2), with and without (control) various other excipients (50 mM concentration).

FIG. 6C is a graph showing the increase in BiTE®-5 aggregation levels after one-month frozen storage at −20° C. in a formulation comprising 10 mM glutamate, 9% sucrose, 0.01% polysorbate 80 (PS80) (pH 4.2), with and without 50 mM tryptophan.

FIG. 7 is a graph showing BiTE®-5 aggregation levels after one-month frozen storage at −15° C. at varying ratios of methionine: BiTE®-5. Storage at −15° C. represents accelerated stress conditions for −30° C. storage. The % HMW, measure using SE-UHPLC (y-axis), is provided for samples at time 0 (dark bars on left) and after four weeks frozen storage (gray bars on right). The ratios are noted on the x-axis.

### DETAILED DESCRIPTION

Despite the many years of research and development devoted to therapeutic antibody products, instability remains an important concern for the industry. Storage and transport of therapeutic antibody formulations are associated with significant challenges, as the conditions must maintain higher-order protein structure while minimizing degradation and aggregation, which negatively impact therapeutic effectiveness and increase potential immunogenicity to the patient. The bispecific antibody constructs described herein, for example, demonstrate reduced stability in frozen form, requiring the use of expensive equipment and inconvenient processes to maintain the formulation at −70° C. Surprisingly, bispecific antibody construct formulations comprising methionine exhibit reduced aggregation upon storage at about −10° C. to about −40° C. (e.g., about −20° C. to about −35° C., such as −30° C.), thereby avoiding the need for equipment and procedures to maintain the therapeutic at much lower temperatures (e.g., −70° C.).

The disclosure provides a pharmaceutical formulation comprising a bispecific antibody construct, a saccharide, a surfactant, a buffer, and methionine. Methionine is optionally present at a molar ratio of methionine to bispecific antibody construct of about 10× to about 5000×. The pH of the formulation is from about 4 to about 7 (such as about 4 to about 6). In various aspects, the formulation is frozen. Alternatively, the formulation is a thawed formulation or is lyophilized. Various aspects of the formulations are described below. The use of section headings is merely for the convenience of reading; it should be understood that all combinations of features described herein are contemplated.

Antibody Constructs

An “antibody construct” is a protein comprising a domain that binds a specified target antigen (such as CD3 and/or CDH19, MSLN, DLL3, FLT3, EGFRvIII, BCMA, PSMA, CD33, CD19, CD70, CLDN18.2, or MUC17). In exemplary aspects, an antibody construct is an antibody or immunoglobulin, an antigen-binding fragment thereof, or an antibody protein product comprising antigen-binding domains in a scaffold, framework, or format that allows an antigen-binding domain to adopt a conformation that promotes binding to the antigen.

The term “antibody” refers to an intact antigen-binding immunoglobulin. The antibody can be an IgA, IgD, IgE, IgG, or IgM antibody, including any one of IgG1, IgG2, IgG3 or IgG4. In various embodiments, an intact antibody comprises two full-length heavy chains and two full-length light chains. An antibody has a variable region and a constant region. In IgG formats, a variable region is generally about 100-110 or more amino acids, comprises three complementarity determining regions (CDRs), is primarily responsible for antigen recognition, and substantially varies among other antibodies that bind to different antigens. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). The constant region allows the antibody to recruit cells and molecules of the immune system.

The architecture of antibodies has been exploited to create a growing range of alternative formats that span a molecular-weight range of at least about 12-150 kDa and have a valency (n) range from monomeric (n=1), to dimeric (n=2), to trimeric (n=3), to tetrameric (n=4), and potentially higher; such alternative formats are referred to herein as “antibody protein products.” Antibody protein products include those based on the full antibody structure and those that mimic antibody fragments which retain full antigen-binding capacity, e.g., scFv, disulfide-bond stabilized scFv (ds-scFv), single chain antibody (SCA), single chain Fab (scFab), and minibodies (miniAbs).

An antibody construct may be “bispecific,” i.e., the antibody or antibody protein product binds two different targets (e.g., CD3 and a second, different target). A “bispecific” antibody or antibody-like product generally comprises a first binding domain and a second binding domain, wherein the first binding domain binds to one antigen or target (e.g., the target cell surface antigen), and the second binding domain binds to another antigen or target (e.g., CD3). Accordingly, the antibody construct optionally comprises specificities for two different antigens or targets. The term “target cell surface antigen” refers to an antigenic structure expressed by a cell and which is present at the cell surface such that it is accessible for an antibody construct as described herein. It may be a protein, preferably the extracellular portion of a protein, or a carbohydrate structure, preferably a carbohydrate structure of a protein, such as a glycoprotein. In various aspects, it is a tumor antigen. Multispecific antibody constructs, such as trispecific antibody constructs (including three binding domains) or constructs having more than three (e.g. four, five, or more) specificities also are contemplated.

Bispecific antibody constructs include, but are not limited to, traditional bispecific immunoglobulins (e.g., BsIgG), IgG comprising an appended antigen-binding domain (e.g., the amino or carboxy termini of light or heavy chains are connected to additional antigen-binding domains, such as single domain antibodies or paired antibody variable domains (e.g., Fv or scFv)), BsAb conjugates, and engineered constructs comprising full length antibodies. See, e.g., Spiess et al., Molecular Immunology 67(2) Part A: 97-106 (2015) and International Patent Publication No. WO 2015149077, which describes various bispecific formats and is hereby incorporated by reference. Examples of bispecific antibody constructs also include, but are not limited to, diabodies, single chain diabodies, tandem scFvs, bispecific T cell engager (BITE®) format (a fusion protein consisting of two single-chain variable fragments (scFvs) joined by a linker), BsAb fragments (e.g., bispecific single chain antibodies), bispecific fusion proteins (e.g., antigen binding domains fused to an effector moiety), and Fab₂ bispecifics (collectively also termed “bispecific antibody protein products”). See, e.g., Chames & Baty, 2009, mAbs 1[6]:1-9; and Holliger & Hudson, 2005, Nature Biotechnology 23[9]:1126-1136; Wu et al., 2007, Nature Biotechnology 25[11]:1290-1297; Michaelson et al., 2009, mAbs 1[2]:128-141; International Patent Publication No. WO 2009032782 and WO 2006020258; Zuo et al., 2000, Protein Engineering 13[5]:361-367; U.S. Patent Application Publication No. 20020103345; Shen et al., 2006, J Biol Chem 281[16]:10706-10714; Lu et al., 2005, J Biol Chem 280[20]:19665-19672; and Kontermann, 2012 MAbs 4(2):182, all of which are expressly incorporated herein.

The term “binding domain” refers to a domain which (specifically) binds to (i.e., interacts with or recognizes) a given target epitope or a given target site on a target molecule (antigen), such as, e.g., CDH19, MSLN, DLL3, FLT3, EGFRvIII, BCMA, PSMA, CD33, CD19, CD70, CLDN18.2, MUC17, or CD3. In a bispecific antibody construct, for example, the structure and function of the first binding domain (recognizing, e.g., CDH19, MSLN, DLL3, FLT3, EGFRvIII, BCMA, PSMA, CD33, CD19, CD70, CLDN18.2, or MUC17), and preferably also the structure and/or function of the second binding domain (recognizing CD3), is/are based on the structure and/or function of an antibody, e.g., of a full-length or whole immunoglobulin molecule. Alternatively, the structure and function are drawn from the variable heavy chain (VH) and/or variable light chain (VL) domains of an antibody or fragment thereof. Preferably, the first binding domain is characterized by the presence of three light chain CDRs (i.e., CDR1, CDR2 and CDR3 of the VL region) and/or three heavy chain CDRs (i.e., CDR1, CDR2 and CDR3 of the VH region). The second binding domain preferably also comprises the minimum structural requirements of an antibody which allow for the target binding. More preferably, the second binding domain comprises at least three light chain CDRs (i.e., CDR1, CDR2 and CDR3 of the VL region) and/or three heavy chain CDRs (i.e., CDR1, CDR2 and CDR3 of the VH region). In various aspects, one or more of the antigen binding domains are human or humanized or chimeric.

In some embodiments, the antibody construct comprises a single chain antibody construct. An scFv comprises a variable heavy chain, an scFv linker, and a variable light domain. Optionally, the C-terminus of the variable light chain is attached to the N-terminus of the scFv linker, the C-terminus of which is attached to the N-terminus of a variable heavy chain (N-vh-linker-vl-C), although the configuration can be switched (N-vl-linker-vh-C). Alternatively, the C-terminus of the variable heavy chain is attached to the N-terminus of the scFv linker, the C-terminus of which is attached to the N-terminus of a variable light chain (N-vl-linker-vh-C), although the configuration can be switched (N-vh-linker-v-C). scFvs in either orientation are contemplated herein, as are scFvs with half-life extending moieties.

Peptide linkers (spacer peptides) may be used in the context of antigen binding domains and variable domains (VH/VL). A peptide linker may link variable domains and/or may be used to fuse a third domain to an antibody construct. Peptide linkers used in the context of the disclosure do not comprise polymerization activity. Peptide linkers also may be used to attach other domains or modules or regions (such as half-life extending domains) to an antigen binding protein, such as the bispecific antibody constructs described herein. Among the suitable peptide linkers are those described in U.S. Pat. Nos. 4,751,180 and 4,935,233 or International Patent Publication No. WO 88/09344, the disclosures of which are incorporated herein by reference in their entireties.

In some embodiments, the antibody construct comprises a third domain comprising a “Fc” or “Fc region” or “Fc domain,” which refers to the polypeptide comprising the constant region of an antibody excluding the first constant region immunoglobulin domain. Thus, “Fc domain” refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cy2 and Cy3 (Cy2 and Cy3) and the lower hinge region between Cy1 (Cy1) and Cy2 (Cy2). A bispecific antibody construct of the disclosure is preferably based on an IgG antibody (which includes several subclasses, including, but not limited to IgG1, IgG2, IgG3, and IgG4). Although the boundaries of the Fc region may vary, the human IgG heavy chain Fc region is usually defined to include residues C226 or P230 to its carboxyl-terminus, wherein the numbering is according to the EU index as in Kabat. In some embodiments, amino acid modifications are made to the Fc region, for example, to alter binding to one or more FcγR receptors or to the FcRn receptor.

In some embodiments, the formulation described herein comprises a bispecific antibody construct comprising a first binding domain that binds to a target cell surface antigen and a second binding domain that binds to human CD3 on the surface of a T cell. In any of the aspects described herein, the target cell surface antigen is CDH19, MSLN, DLL3, FLT3, EGFR, EGFRvIII, BCMA, PSMA, CD33, CD19, CD70, MUC17 or CLDN18.2. The bispecific antibody construct, in various aspects, comprises a third domain comprising, in an amino to carboxyl order, hinge-CH2 domain-CH3 domain-linker-hinge-CH2 domain-CH3 domain. In some embodiments, each of the first and second binding domains comprise a VH region and a VL region. Thus, in some embodiments, the formulations described herein comprise a bispecific antibody construct which binds human CD3 and human CDH19, or human CD3 and human MSLN, or human CD3 and human DLL3, or human CD3 and human FLT3, or human CD3 and human EGFRvIII, or human CD3 and human BCMA, or human CD3 and PSMA, or human CD3 and human CD33, or human CD3 and human CD19, human CD3 and human CD70, or human CD3 and human MUC17, or human CD3 and human CLDN18.2.

In some embodiments, the first binding domain of the bispecific antibody construct comprises a set of six CDRs set forth in (a) SEQ ID NOs: 24-29, (b) SEQ ID NOs: 34-39, (c) SEQ ID NOs: 78-83, (d) SEQ ID NOs: 10-15, (e) SEQ ID NOs: 46-51, (f) SEQ ID NOs: 88-93, (g) SEQ ID NOs: 67-72, (h) SEQ ID NOs: 56-61, (i) SEQ ID NOs: 112-117, (j) SEQ ID NOs: 100-105, (k) SEQ ID NOs:148-153, SEQ ID NOs: 157-162, or SEQ ID NOs: 166-171, or SEQ ID NOs: 175-180, (l) SEQ ID NOs:132-137, or (m) SEQ ID NOs: 123-128.

In some embodiments, the first binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 30, 40, 84, 16, 17, 52, 94, 73, 62, 118, 154, 163, 172, 181, 106, 138, 143, or 129. In some embodiments, the first binding domain of the bispecific antibody construct comprises a VH comprising the amino acid sequence set forth in SEQ ID NO: 30, 40, 84, 16, 17, 52, 94, 73, 62, 118, 154, 163, 172, 181, 106, 138, 143, or 129.

In some embodiments, the first binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 31, 41, 85, 18, 19, 53, 95, 74, 63, 119, 155, 164, 173, 182, 107, 139, 144, or 130. In some embodiments, the first binding domain of the bispecific antibody construct comprises a VL comprising the amino acid sequence set forth in SEQ ID NO: 31, 41, 85, 18, 19, 53, 95, 74, 63, 119, 155, 164, 173, 182, 107, 139, 144, or 130.

In some embodiments, the first binding domain comprises (a) a VH region comprising an amino acid sequence set forth in SEQ ID NO: 30 and a VL region comprising an amino acid sequence set forth in SEQ ID NO: 31; (b) a VH region comprising an amino acid sequence set forth in SEQ ID NO: 40 and a VL region comprising an amino acid sequence set forth in SEQ ID NO: 41; (c) a VH region comprising an amino acid sequence set forth in SEQ ID NO: 84 and a VL region comprising an amino acid sequence set forth in SEQ ID NO: 85; (d) a VH region comprising an amino acid sequence set forth in SEQ ID NO: 16 or 17 and a VL region comprising an amino acid sequence set forth in SEQ ID NO: 18 or 19; (e) a VH region comprising an amino acid sequence set forth in SEQ ID NO: 52 and a VL region comprising an amino acid sequence set forth in SEQ ID NO: 53; (f) a VH region comprising an amino acid sequence set forth in SEQ ID NO: 94 and a VL region comprising an amino acid sequence set forth in SEQ ID NO: 95; (g) a VH region comprising an amino acid sequence set forth in SEQ ID NO: 73 and a VL region comprising an amino acid sequence set forth in SEQ ID NO: 74; (h) a VH region comprising an amino acid sequence set forth in SEQ ID NO: 62 and a VL region comprising an amino acid sequence set forth in SEQ ID NO: 63; (i) a VH region comprising an amino acid sequence set forth in SEQ ID NO: 118 and a VL region comprising an amino acid sequence set forth in SEQ ID NO: 119; (j) a VH region comprising an amino acid sequence set forth in SEQ ID NO: 154, 163, 172 or 181 and a VL region comprising an amino acid sequence set forth in SEQ ID NO: 155, 164, 173 or 182; (k) a VH region comprising an amino acid sequence set forth in SEQ ID NO: 106 and a VL region comprising an amino acid sequence set forth in SEQ ID NO: 107; (l) a VH region comprising an amino acid sequence set forth in SEQ ID NO: 138 or 143 and a VL region comprising an amino acid sequence set forth in SEQ ID NO: 139 or 144; or (m) a VH region comprising an amino acid sequence set forth in SEQ ID NO: 129 and a VL region comprising an amino acid sequence set forth in SEQ ID NO: 130.

In some embodiments, the second binding domain of the bispecific antibody construct comprises a set of six CDRs set forth in SEQ ID NOs: 1-6.

In some embodiments, the second binding domain of the bispecific antibody construct comprises a VH region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 7. In some embodiments, the second binding domain of the bispecific antibody construct comprises a VH comprising the amino acid sequence set forth in SEQ ID NO: 7.

In some embodiments, the second binding domain of the bispecific antibody construct comprises a VL region comprising an amino acid sequence at least 90% identical (e.g., 91%, 92%, 93%, 94%, 95%, 96%, 97%, 98%, 99%, or 100% identical) to the amino acid sequence set forth in SEQ ID NO: 8. In some embodiments, the second binding domain of the bispecific antibody construct comprises a VL comprising the amino acid sequence set forth in SEQ ID NO: 8.

In some embodiments, the second binding domain comprises a VH region comprising an amino acid sequence set forth in SEQ ID NO: 7 and a VL region comprising an amino acid sequence set forth in SEQ ID NO: 8.

In some embodiments, the bispecific antibody construct comprises a first binding domain that binds CD19 comprising an anti-CD19 variable light domain comprising the amino acid sequence of SEQ ID NO: 85 and an anti-CD19 variable heavy domain comprising the amino acid sequence of SEQ ID NO: 84, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 86 and a second binding domain comprising the amino acid sequence of SEQ ID NO: 9. In some embodiments, the bispecific antibody construct comprises the amino acid sequence set forth in SEQ ID NO: 87.

In some embodiments, the bispecific antibody construct comprises a first binding domain that binds MSLN comprising an anti-MSLN variable light domain comprising the amino acid sequence of SEQ ID NO: 41 and an anti-MSLN variable heavy domain comprising the amino acid sequence of SEQ ID NO: 40, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 42 and a second binding domain comprising the amino acid sequence of SEQ ID NO: 9. In some embodiments, the bispecific antibody construct comprises an amino acid sequence set forth in SEQ ID NO: 43, 44, or 45.

In some embodiments, the bispecific antibody construct comprises a first binding domain that binds DLL3 comprising an anti-DLL3 variable light domain comprising the amino acid sequence of SEQ ID NO: 74 and an anti-DLL3 variable heavy domain comprising the amino acid sequence of SEQ ID NO: 73, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 75 and a second binding domain comprising the amino acid sequence of SEQ ID NO: 9. In some embodiments, the bispecific antibody construct comprises an amino acid sequence set forth in SEQ ID NO: 76 or 77.

In some embodiments, the bispecific antibody construct comprises a first binding domain that binds FLT3 comprising an anti-FLT3 variable light domain comprising the amino acid sequence of SEQ ID NO: 63 and an anti-FLT3 variable heavy domain comprising the amino acid sequence of SEQ ID NO: 62, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 64 and a second binding domain comprising the amino acid sequence of SEQ ID NO: 9. In some embodiments, the bispecific antibody construct comprises an amino acid sequence set forth in SEQ ID NO: 65 or 66.

In some embodiments, the bispecific antibody construct comprises a first binding domain that binds EGFRvIII comprising an anti-EGFRvIII variable light domain comprising the amino acid sequence of SEQ ID NO: 31 and an anti-EGFRvIII variable heavy domain comprising the amino acid sequence of SEQ ID NO: 30, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 32 and a second binding domain comprising the amino acid sequence of SEQ ID NO: 9. In some embodiments, the bispecific antibody construct comprises an amino acid sequence set forth in SEQ ID NO: 33.

In some embodiments, the bispecific antibody construct comprises a first binding domain that binds BCMA comprising an anti-BCMA variable light domain comprising the amino acid sequence of SEQ ID NO: 95 and an anti-BCMA variable heavy domain comprising the amino acid sequence of SEQ ID NO: 94, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 96 and a second binding domain comprising the amino acid sequence of SEQ ID NO: 9. In some embodiments, the bispecific antibody construct comprises an amino acid sequence set forth in SEQ ID NO: 98 or SEQ ID NO: 97.

In some embodiments, the bispecific antibody construct comprises a first binding domain that binds PSMA comprising an anti-PSMA variable light domain comprising the amino acid sequence of SEQ ID NO: 119 or 107 and an anti-PSMA variable heavy domain comprising the amino acid sequence of SEQ ID NO: 118 or 106, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in an embodiment, the bispecific antibody construct comprises a first binding domain comprising the amino acid sequence of SEQ ID NO: 120 or 108 and a second binding domain comprising the amino acid sequence of SEQ ID NO: 9. In some embodiments, the bispecific antibody construct comprises an amino acid sequence set forth in SEQ ID NO: 121, 122, 109, 110, or 111.

In some embodiments, the bispecific antibody construct comprises a first binding domain that binds CD33 comprising an anti-CD33 variable light domain comprising the amino acid sequence of SEQ ID NO: 18 or 19 and an anti-CD33 variable heavy domain comprising the amino acid sequence of SEQ ID NO: 16 or 17, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 189 or 190 and a second binding domain comprising the amino acid sequence of SEQ ID NO: 9. In some embodiments, the bispecific antibody construct comprises the amino acid sequence set forth in SEQ ID NO: 20, 21, 22, or 23.

In some embodiments, the bispecific antibody construct comprises a first binding domain that binds CDH19 comprising an anti-CDH19 variable light domain comprising the amino acid sequence of SEQ ID NO: 53 and an anti-CDH19 variable heavy domain comprising the amino acid sequence of SEQ ID NO: 52, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 54 and a second binding domain comprising the amino acid sequence of SEQ ID NO: 9. In some embodiments, the bispecific antibody construct comprises the amino acid sequence set forth in SEQ ID NO: 55.

In some embodiments, the bispecific antibody construct comprises a first binding domain that binds MUC17 comprising an anti-MUC17 variable light domain comprising the amino acid sequence of SEQ ID NO: 155, 164, 173, or 182 and an anti-MUC17 variable heavy domain comprising the amino acid sequence of SEQ ID NO: 154, 163, 172, or 181, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in embodiments, the bispecific antibody construct comprises a first binding domain comprising the amino acid sequence of SEQ ID NO: 194 and a second binding domain comprising the amino acid sequence set forth in SEQ ID NO: 195 (optionally with an Fc domain comprising the amino acid sequence of SEQ ID NO: 196). In some embodiments, the bispecific antibody construct comprises the amino acid sequence set forth in SEQ ID NO: 156, 165, 174 or 183.

In some embodiments, the bispecific antibody construct comprises a first binding domain that binds cldn18.2 comprising an anti-cldn18.2 variable light domain comprising the amino acid sequence of SEQ ID NO: 139 or 144 and an anti-cldn18.2 variable heavy domain comprising the amino acid sequence of SEQ ID NO: 138 or 143, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in one embodiment, the bispecific antibody construct comprises a first binding domain comprising the amino acid sequence of SEQ ID NO: 140 or 145 and a second binding domain comprising the amino acid sequence of SEQ ID NO: 9. In some embodiments, the bispecific antibody construct comprises the amino acid sequence set forth in SEQ ID NO: 141, 142, 146 or 147.

In some embodiments, the bispecific antibody construct comprises a first binding domain that binds CD70 comprising an anti-CD70 variable light domain comprising the amino acid sequence of SEQ ID NO: 130 and an anti-CD70 variable heavy domain comprising the amino acid sequence of SEQ ID NO: 129, a second binding domain comprising an anti-CD3 variable heavy domain comprising the amino acid sequence of SEQ ID NO: 7, and an anti-CD3 variable light domain comprising the amino acid sequence of SEQ ID NO: 8. For example, in embodiments, the bispecific antibody construct comprises a first binding domain comprising the amino acid sequence of SEQ ID NO: 191 and a second binding domain comprising the amino acid sequence set forth in SEQ ID NO: 192 (optionally with an Fc domain comprising the amino acid sequence of SEQ ID NO: 193). In some embodiments, the bispecific antibody construct comprises an amino acid sequence set forth in SEQ ID NO: 131.

In some embodiments, the formulation comprises an antibody construct (e.g., bispecific antibody construct) in a concentration ranging from about 1 mg/mL to about 20 mg/mL (e.g., from about 1 mg/mL to about 8 mg/mL, or from about 1 mg/mL to about 5 mg/mL). In some embodiments, the formulation comprises an antibody construct (e.g., a bispecific antibody construct) in a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL or about 20 mg/mL.

In some embodiments, the disclosure provides a formulation comprising a bispecific antibody construct that co-engages human T cell CD3 and one of human CDH19, human MSLN, human DLL3, human FLT3, human EGFRvIII, human BCMA, human PSMA, human CD33, human CD19, human CD70, human CLDN18.2 or human MUC17, in such a manner so as to transiently connect malignant cells with T cells, thereby inducing T cell mediated killing of the bound malignant cell. The formulation preferably comprises about 1-20 mg/mL of bispecific antibody construct, a buffer, a saccharide, a surfactant, and methionine present at a molar ratio of methionine to bispecific antibody construct of about 10× to about 5000×, wherein the formulation has a pH ranging from about 4-7 (e.g., about 4-6, such as about 4.2).

Buffers

Buffering agents are often employed to control pH in the formulation. The formulation of the disclosure comprises a buffer, which optionally may be an acetate buffer, a glutamate buffer, a citrate buffer, a lactic buffer, a succinate buffer, a tartrate buffer, a fumarate buffer, a maleate buffer, a histidine buffer, or a phosphate buffer (or a combination thereof). In various embodiments, the buffer is a glutamate buffer. Optionally, the pH of the formulation is about 4 to about 7 (e.g., about 4 to about 6, such as about 4.2).

The buffer may be present in any amount suitable to maintain the pH of the formulation at a predetermined level. The buffer may be present at a concentration between about 0.1 mM and about 1000 mM (1 M), or between about 5 mM and about 200 mM, or between about 5 mM to about 100 mM, or between about 10 mM and about 50 mM. Suitable buffer concentrations encompass concentrations of about 200 mM or less. In some embodiments, the buffer in the formulation is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM, or about 5 mM. In some embodiments, the concentration of the buffer is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some embodiments, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some embodiments, the concentration of the buffer is between 5, 6, 7, 8, 9, 10, 11, 12, 13, 14, 15, 16, 17, 18, 19, 20, 30, or 40 mM and 50 mM. In some embodiments, the concentration of the buffer (e.g., glutamate buffer) is about 10 mM.

Surfactants

The formulation described herein comprises, in various embodiments, a surfactant. Optionally, the surfactant is a nonionic surfactant. Exemplary surfactants include but are not limited to, polysorbate 20, polysorbate 40, polysorbate 60, polysorbate 80, poloxamer 188, poloxamer 407, Triton™ X-100, polyoxyethylene, PEG 3350, PEG 4000, or a combination thereof. In various aspects, the surfactant is polysorbate 20, polysorbate 40, polysorbate 60, or polysorbate 80. In an exemplary embodiment, the surfactant is polysorbate 80.

Various formulations described herein comprise at least one surfactant, either individually or as a mixture in different ratios. In some embodiments, a surfactant is included at a concentration of about 0.001% to about 5% w/v (or about 0.001% to about 0.5%, or about 0.004 to about 0.5% w/v). In some embodiments, the formulation comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some embodiments, the formulation comprises a surfactant at a concentration of about 0.001% to about 0.5% w/v (e.g., about 0.001 to about 0.01% w/v). In some embodiments, the formulation comprises a surfactant at a concentration of about 0.001%, about 0.002%, about 0.003%, about 0.004%, about 0.005%, about 0.006%, about 0.007%, about 0.008%, about 0.009%, about 0.01%, about 0.05%, about 0.1%, about 0.2%, about 0.3%, about 0.4%, to about 0.5% w/v. In some embodiments, the formulation comprises a surfactant incorporated in a concentration of about 0.001% to about 0.01% w/v. In some embodiments, the surfactant is polysorbate 80 and the polysorbate 80 is present in a concentration of about 0.01% w/v.

Saccharides

The formulation described herein comprises a saccharide. In some embodiments, the saccharide is a monosaccharide or a disaccharide. In some embodiments, the saccharide is glucose, galactose, fructose, xylose, sucrose, lactose, maltose, trehalose, sorbitol, mannitol or xylitol, or a combination thereof.

In some embodiments, the formulation comprises a saccharide at a concentration of about 0.01% to about 40% w/v, or about 00.1% to about 20% w/v, or about 1% to about 15%, or about 5% to about 12%, or about 7% to about 12% w/v. In some embodiments, the formulation comprises at least one saccharide at a concentration of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, or at least 40% w/v. In some embodiments, the formulation comprises at least one saccharide at a concentration of about 1%, about 2%, about 3%, about 4%, about 5%, about 6%, about 7%, about 8%, about 9%, about 10%, about 11%, about 12%, about 13%, about 14%, or about 15% w/v. In a yet further embodiment, the pharmaceutical formulation comprises at least one saccharide at a concentration of about 7%, about 7.5%, about 8%, about 8.5%, about 9%, about 9.5%, about 10%, about 10.5%, about 11%, about 11.5%, or about 12% w/v. In some embodiments, the pharmaceutical formulation comprises at least one saccharide at a concentration of about 7% to about 12% w/v. In some embodiments, a saccharide (e.g. sucrose) is in the formulation at a concentration of about 9% w/v.

Methionine

The formulation of the disclosure comprises methionine. Methionine is present at a molar ratio of methionine to bispecific antibody construct of about 5× to about 5000×, such as about 5× to about 4200×, about 10× to about 5000×, or about 10× to about 4200×. In various aspects, methionine is present at a molar ratio of methionine to bispecific antibody construct of about 50× (e.g., 105×) to about 5000×. For example, the methionine may be present at a molar ratio of methionine to bispecific antibody construct of about 100× to about 4500×, about 5× to about 1000×, about 10× to about 2500×, about 100× to about 1500×, about 200× to about 2500×, or about 500× to about 1500×. The methionine may be present at a molar ratio of methionine to bispecific antibody construct of greater than 5×, 10×, 20×, 100×, 105×, 200×, 500×, 1000×, 2000×, 4000×, 4200×, 4500×, or 5000× (e.g., greater than or equal to any of these values). The methionine may be present at a molar ratio of methionine to bispecific antibody construct of no more than 5×, 10×, 20×, 100×, 200×, 500×, 1000×, 2000×, 4000×, 4200×, 4500× or 5000×. For example, in an exemplary aspect of the disclosure, the ratio of methionine to bispecific antibody construct is at least about 105×, and the bispecific antibody construct optionally comprises CDR sequences of SEQ ID NOs: 67-72 and SEQ ID NOs: 1-6 or the amino acid sequence of SEQ ID NO: 77. Methods of determining a molar ratio are well understand in the art; for example, when a bispecific antibody construct is provided at a concentration of 2.5 mg/mL, 0.125 mM methionine results in a molar ratio of 5.25, 0.25 mM methionine results in a molar ratio of 10.5, 0.5 mM methionine results in a molar ratio of 21, 2.5 mM methionine results in a molar ratio of 105, 5 mM methionine results in a molar ratio of 210, 12.5 mM methionine results in a molar ratio of 525, 25 mM methionine results in a molar ratio of 1050, 50 mM methionine results in a molar ratio of 2100, and 100 mM methionine results in a molar ratio of 4200. In various aspects, the formulation comprises about 10 mM to about 200 mM (e.g., about 20 mM to about 150 mM, about 25 mM to about 75 mM, or about 50 mM to about 100 mM) methionine. In various aspects, the formulation comprises about 50 mM methionine.

The term “aggregate” generally refers to protein species of higher molecular weight (HMW), instead of the desired defined species (e.g., a monomer). The term is used interchangeably herein with the terms “high molecular weight species” and “HMW” (i.e., molecules having a higher molecular weight than pure product molecules). Aggregates may generally differ in size (ranging from small (dimers) to large assemblies (subvisible or even visible particles) and from the nanometer to micrometer range in diameter), morphology (approximately spherical to fibrillar), protein structure (native vs. non-native/denatured), type of intermolecular bonding (covalent vs. non-covalent), reversibility, and solubility. Soluble aggregates cover the size range of roughly 1 to 100 nm, and protein particulates cover subvisible (˜0.1-100 nm) and visible (>100 nm) ranges. The term “aggregate” refers to all kinds physically-associated or chemically linked non-native species of two or more protein monomers, including amorphous aggregates, oligomers, multimers, and the like. The term “aggregation” refers to the direct mutual attraction between molecules, e.g., via van der Waals forces or chemical bonding.

As described in the Example, the addition of methionine to a formulation comprising a bispecific antibody construct allows for storage of the formulation as a frozen formulation at about −10° C. to about −40° C. (e.g., at about −20° C. to about −35° C. or about 30° C.) without incurring the level of aggregation encountered when methionine is lacking from the formulation. The stability of a bispecific antibody construct formulation can be quantified in several ways, including size exclusion high performance liquid chromatography (SE-HPLC), size exclusion ultra high performance liquid chromatography (SE-UHPLC), cation exchange high performance liquid chromatography (CE-H PLC), dynamic light scattering, analytical ultracentrifugation (AUC), field flow fractionation (FFF), isoelectric focusing, and ion exchange chromatography (IEX). A preferred method of determining the presence of HMW species in a bispecific antibody construct formulation is SE-UHPLC. Exemplary conditions for conducting SE-UHPLC are provided in the Example. For example, the formation of HMW species or the rate of increase of the amount of HMW species of the bispecific antibody construct may be determined at various time points. For instance, the amount of HMW species may be determined at one week, two weeks, four weeks, three months, six months, twelve months, eighteen months or two years in storage at approximately −10° C. to −40° C. (e.g., −15° C., which represents accelerated stress conditions for −30° C. storage).

In some embodiments, the relative values of any particular species of the bispecific antibody construct, such as the intact BiTE® molecule or main species, or the high molecular weight (HMW) species (i.e., aggregates), are expressed in relation to the respective values of the total product. For example, in some embodiments, 10% or less (e.g., 10%, 9%, 8%, 7%, 6%, 5%, 4%, 3.5%, 3%, 2.5%, 2%, 1.5%, 1%, 0.5%, or less) of the bispecific antibody construct exists as HMW species in the formulation after storage for a particular length of time (e.g., four weeks) at −10° C. to −40° C., such as −20° C. to −35° C. (e.g., −30° C. or −15° C.). Alternatively, the relative values of a particular species in different formulations stored under similar conditions may be compared. For example, the formulation comprising methionine comprises at least about 10% less HMW species compared to a matched formulation not comprising methionine stored under the same conditions for the same time period. In various aspects, the formulation comprising methionine comprises at least about 25% to about 85% less HMW species after storage at −10° C. to −40° C. (e.g., −20° C. to −35° C., −30° C., or −15° C.) for a period of time (e.g., four weeks) compared to a matched formulation not comprising methionine stored under the same conditions for the same time period. A “matched formulation” is a formulation comprising the same components in the same amounts but lacking methionine. In various aspects, the formulation of the disclosure comprises about 30% to about 75% less HMW species (e.g., about 25% to about 60% less, or about 30% to about 60% less HMW species) after storage at −10° C. to −40° C. (e.g., −20° C. to −35° C., or −15° C. or −30° C.) for a period of time (e.g., four weeks) compared to a matched formulation not comprising methionine.

Thus, in various aspects, the disclosure provides a formulation comprising about 1 mg/mL to about 20 mg/mL bispecific antibody construct, 10 mM glutamate, 9% sucrose, 0.01% PS80, 50 mM methionine at pH 4.2. The disclosure also provides a frozen pharmaceutical formulation comprising about 1 mg/mL to about 20 mg/mL bispecific antibody construct, sucrose (e.g., about 9% sucrose), glutamic acid (e.g., about 10 mM glutamic acid), polysorbate 80 (e.g., about 0.01% PS80), and about 10 mM to about 200 mM methionine, wherein the pH of the formulation is from about 4 to about 7 (e.g., about 4 to about 6, such as about 4.2).

Therapeutic Use of the Formulation

The formulation described herein is useful as a pharmaceutical formulation in the treatment or amelioration of cancer in a subject in need thereof. The terms “subject in need” or those “in need of treatment” include subjects already afflicted with the disorder, as well as those in which the disorder is to be prevented. The “subject in need” or “patient” includes human and other mammalian subjects that receive either prophylactic or therapeutic treatment. The term “treatment” refers to both therapeutic treatment and prophylactic or preventative measures. “Treatment” does not require complete remission or eradication of the disease; any improvement in the disease and/or improvement in the symptoms associated with the disease are contemplated. For example, a therapeutic response would refer to one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) slowing of disease progression; (6) an increased patient survival rate; (7) downgrade of stage of a cancer (e.g., Stage 2 to Stage 1) and/or (8) some relief from one or more symptoms associated with the disease or condition. “Prevention” includes, e.g., the avoidance of an occurrence or re-occurrence a tumor or cancer. Disease state is monitored by, e.g., clinical examination, X-ray, computerized tomography (CT, such as spiral CT), magnetic resonance imaging (MRI), positron emission tomography (PET), ultrasound, endoscopy and laparoscopy, tumor marker levels (e.g., carcinoembryonic antigen (CEA)), cytology, histology, tumor biopsy sampling, and/or counting of tumor cells in circulation. These methods also are typically used to diagnose and stage cancer.

The disclosure provides a method of treating cancer, comprising administering to a subject in need thereof a therapeutically effective amount of the formulation described herein. In certain embodiments, the subject is a human. In certain embodiments, the cancer is a solid tumor. In some embodiments, the cancer is brain cancer, bladder cancer, breast cancer (e.g., triple negative breast cancer), clear cell kidney cancer, cervical cancer, colon and rectal cancer, endometrial cancer, gastric cancer, head/neck squamous cell carcinoma, lip and oral cancer, liver cancer, lung squamous cell carcinoma, melanoma, mesothelioma, non-small-cell lung cancer (NSCLC), non-melanoma skin cancer, ovarian cancer, oral cancer, pancreatic cancer, prostate cancer, neuroendocrine prostate cancer, renal cell carcinoma, sarcoma, small-cell lung cancer (SCLC), Squamous Cell Carcinoma of the Head and Neck (SCCHN), or thyroid cancer.

In some embodiments, the cancer is adrenocortical tumor, alveolar soft part sarcoma, carcinoma, chondrosarcoma, colorectal carcinoma, desmoid tumor, desmoplastic small round cell tumor, endocrine tumor, endodermal sinus tumor, epithelioid hemangioendothelioma, Ewing sarcoma, germ cell tumor, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.

In some embodiments, the cancer is acute lymphoblastic leukemia (ALL), acute myeloid leukemia (AML), chronic lymphocytic leukemia (CLL), or chronic myeloid leukemia (CML).

In some embodiments, the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL).

Cancers that can be treated include, but are not limited to, alveolar rhabdomyosarcoma, bone cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, esophageal cancer, gastrointestinal carcinoid tumor, hypopharynx cancer, larynx cancer, nasopharynx cancer, peritoneum, omentum, and mesentery cancer, pharynx cancer, small intestine cancer, neuroendocrine cancer, soft tissue cancer, stomach cancer, testicular cancer, ureter cancer, and urinary bladder cancer.

Routes of Administration

Preferably, the pharmaceutical formulation is administered parenterally, e.g., intravenously, subcutaneously, intratumorally, or intramuscularly. Parenteral administration may be achieved by injection, such as bolus injection, or by infusion, such as continuous infusion. Administration may be achieved via depot for long-term release. In some embodiments, the formulation is administered intravenously by an initial bolus followed by a continuous infusion to maintain therapeutic circulating levels of drug product. In some embodiments, the formulation is administered as a one-time dose. Pharmaceutical formulations may be administered using a medical device. Examples of medical devices for administering pharmaceutical formulations are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

In various aspects, the formulation is frozen, and the method comprises thawing the formulation prior to administration to the subject. In alternative aspects, the formulation is lyophilized, and the formulation is reconstituted with an appropriate diluent. In these and other aspects, the resulting formulation (thawed or reconstituted) is administered intravenously.

Other Methods

The disclosure further provides a method comprising (a) preparing a formulation comprising a bispecific antibody construct, methionine, and a buffer, wherein the methionine is present at a molar ratio of methionine to bispecific antibody construct of about 10× to about 5000× (e.g., about 50× to about 5000×); (b) freezing the formulation of (a); and (c) storing the formulation of (b) at a temperature of about −10° C. to about −40° C. The formulation of (a), in some embodiments, further comprises a saccharide and comprises pH of from about 4 to about 7 (e.g., about 4 to about 6). Optionally, steps (b) and (c) are performed at a temperature of about −20° C. to about −35° C. (e.g., about −30° C.) and/or step (c) comprises storing the formulation for at least one month. Also optionally, the method further comprises (d) thawing the formulation of (c); and (e) lyophilizing the formulation of (d). Methionine need not be removed during any of the process steps described herein. In some aspects of the disclosure, a buffer exchange step is performed between step (d) and (e) to produce a pharmaceutical formulation comprising the bispecific antibody construct, a saccharide, a surfactant, a buffer, and methionine present at a molar ratio of methionine to bispecific antibody construct of about 10× to about 5000× (e.g., about 50× to about 5000×), wherein the pH of the formulation is from about 4 to about 7 (e.g., about 4 to about 6, such as about 4.2). Exemplary methods for buffer exchange are known in the art, including dialysis, ultrafiltration and diafiltration, gel filtration and size exchange chromatography. Alternatively, in some aspects of the disclosure, a buffer exchange step is performed between step (d) and (e) to remove the methionine from the formulation, resulting in a pharmaceutical formulation comprising the bispecific antibody construct, a saccharide, a surfactant, and a buffer, wherein the pH of the formulation is from about 4 to about 7 (e.g., about 4 to about 6, or about 4.2). In various aspects, the formulation comprises about 10 mM to about 200 mM methionine, and optionally comprises about 1 mg/ml to about 20 mg/ml bispecific antibody construct.

All of the features described herein with respect to the formulation of the disclosure also apply to the method. For example, the saccharide may be a monosaccharide or a disaccharide, and may be selected from glucose, galactose, fructose, xylose, sucrose, lactose, maltose, trehalose, sorbitol, mannitol, or xylitol. The surfactant may be selected from polysorbate 20, polysorbate 40, polysorbate 60, polysorbate 80, poloxamer 188, poloxamer 407, or Triton™ x-100. The buffer may be selected from an acetate buffer, a glutamate buffer, a citrate buffer, a lactic buffer, a succinate buffer, a tartrate buffer, a fumarate buffer, a maleate buffer, a histidine buffer, or a phosphate buffer. The bispecific antibody construct may be any of the bispecific antibody constructs described herein, such as a bispecific antibody construct comprising the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 55, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

Kits

As an additional aspect, provided herein are kits which comprise a formulation described herein packaged in a manner which facilitates its use for administration to subjects. In one embodiment, such a kit includes a formulation described herein (e.g., a formulation comprising a bispecific antibody construct described therein), packaged in a container such as a sealed bottle, vessel, single-use or multi-use vial, prefilled syringe, or prefilled injection device, optionally with a label affixed to the container or included in the package that describes use of the formulation in practicing the method. In one aspect, the formulation is packaged in a unit dosage form. The kit may further include a device suitable for administering the formulation according to a specific route of administration. Preferably, the kit contains a label that describes use of the formulation described herein.

The invention is further described in the following example. The example serves only to illustrate the invention and is not intended to limit the scope of the invention in any way.

### EXAMPLES

### Example 1

The following Example demonstrates the stability of the bispecific antibody construct formulation of the disclosure after storage at −10° C. to −40° C. (e.g., −15° C.) at four weeks.

Compositions comprising 10 mM glutamate, 9% sucrose, 0.01% PS80, 50 mM methionine, pH 4.2, were prepared, each comprising one of the following bispecific antibody constructs: BiTE®-1 (PSMA×CD3), BiTE®-2 (MSLN×CD3), BiTE®-3 (CD19×CD3), BiTE®-4 (CD33×CD3), BiTE®-5 (DLL3×CD3), BiTE®-6 (FLT3×CD3), BiTE®-7 (BCMA×CD3), and BiTE®-8 (CLDN18.2×CD3). The final protein concentration for each of BiTE®-1, BiTE®-2, BiTE®-3, BiTE®-4, BiTE®-6, BiTE®-7, and BiTE®-8 in their respective compositions was 1.5 mg/mL. The final protein concentration for BiTE®-5 was 3.75 mg/mL.

Protein samples were staged at −20° C. for 24 hours to ensure complete freezing. The samples were then stored at −15° C. for four weeks. In parallel, additional samples were stored at 4° C. and 40° C. to characterize the liquid stability of the formulation with methionine. Some samples were lyophilized to assess the impact of methionine on the lyophilized cake. Lyophilized samples were stored at 4° C. and 40° C.

The time 0 and stressed samples were evaluated for HMW content by Size Exclusion Ultra High-Performance Liquid Chromatography (SE-UHPLC). SE-UHPLC separates proteins based on differences in their hydrodynamic volumes. Molecules with higher hydrodynamic volumes elute earlier than molecules with smaller volumes. The samples were loaded onto an SE-UHPLC column (BEH200, 4.6×300 mm (Waters Corporation, 186005226)), separated isocratically, and the eluent monitored by UV absorbance. Purity was determined by calculating the percentage of each separated component as compared to the total integrated area. SE-UHPLC settings were as follows: Flow rate: 0.4 mL/min; Run time: 12 min; UV detection: 280 nm; Column temperature: Ambient; Target protein load: 6 μg; Protein compatible flow cell: 5 mm.

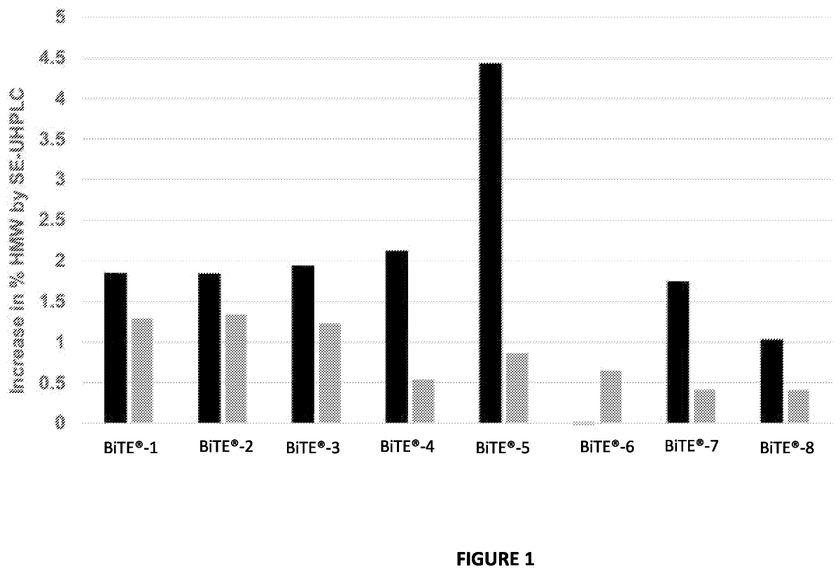

As shown in FIG. 1, the addition of methionine reduced frozen state aggregation levels for various bispecific antibody constructs tested after one month storage at −15° C., which represents accelerated test conditions for −30° C. storage. In a representative experiment, addition of methionine reduced the appearance of HMW species by about 25% to about 85%: BiTE®-1 HMW species reduced about 30%, BiTE®-2 HMW species reduced about 27%, BiTE®-3 HMW species reduced about 36%, BiTE®-4 HMW species reduced about 75%, BiTE®-5 HMW species reduced about 80%, BiTE®-7 HMW species reduced about 76%, and BiTE®-8 HMW species reduced about 60%.

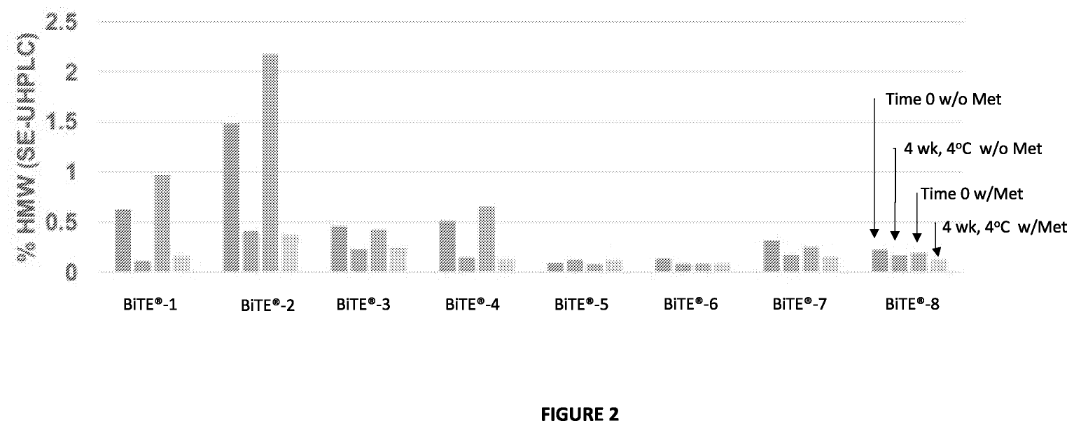

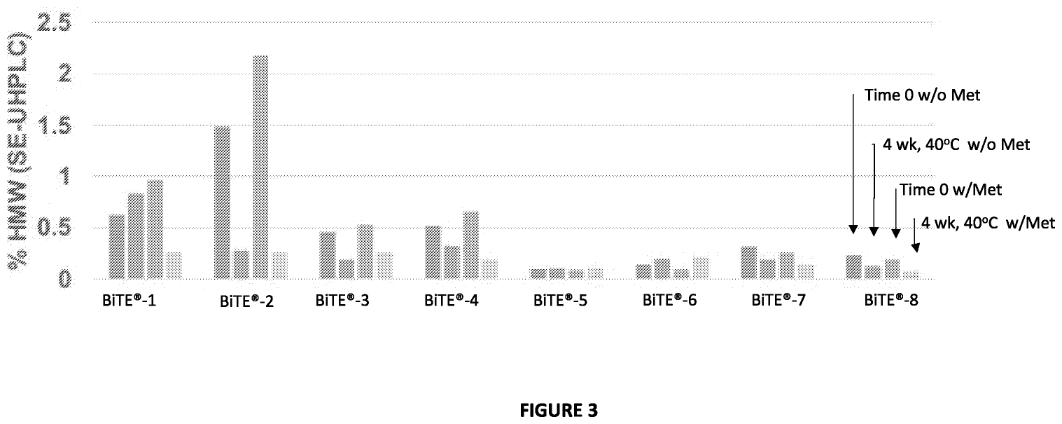

Methionine's inhibitory effect on aggregation on frozen compositions was surprising, at least in part, because methionine did not display a similar effect on liquid compositions. The impact of methionine on liquid stability was assessed after four weeks' storage at 4° C. and 40° C., and it was determined that the excipient did not impact the liquid stability of the bispecific antibody constructs tested. See FIG. 2 and FIG. 3. The percent HMW species detected in samples stored for four weeks at 4° C. was relatively unaffected by the presence of methionine in the formulation (compare the second and fourth bars in FIG. 2). Similar results were observed under accelerated storage conditions of four weeks at 40° C. (compare the second and fourth bars in FIG. 3).

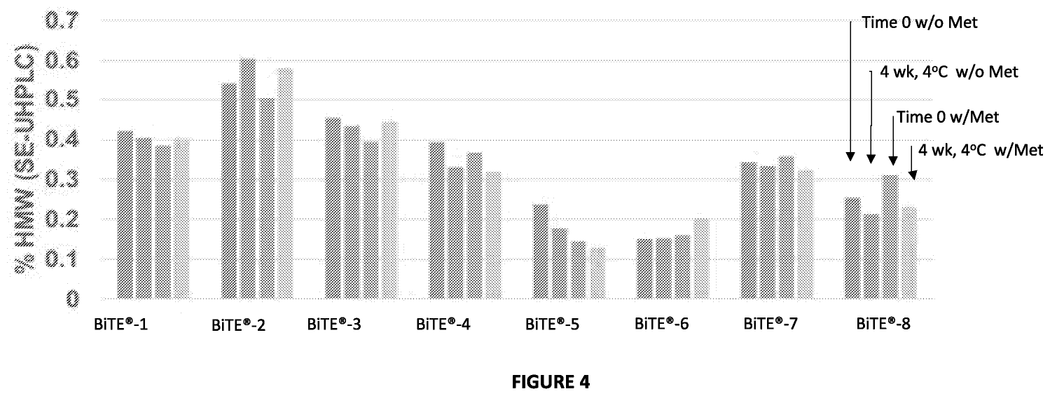

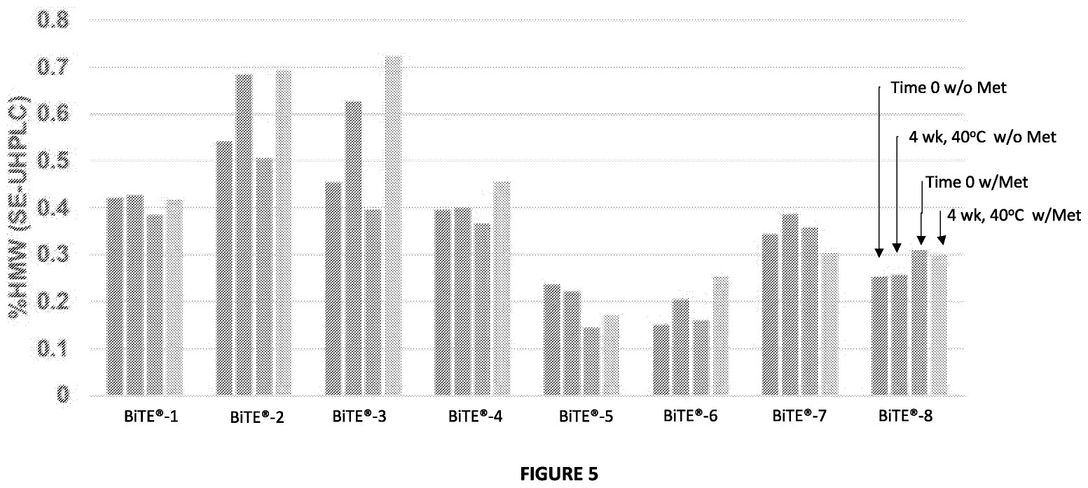

In some circumstances, therapeutic protein compositions are lyophilized for storage or transport. The impact of methionine on lyophilized stability was assessed after storage for four weeks at 4° C. and 40° C. See FIGS. 4 and 5. The higher temperature represents an accelerated stability condition. It was determined that methionine did not impact the lyophilized stability of the bispecific antibody constructs tested (compare the second and fourth bars for each construct in FIGS. 4 and 5).

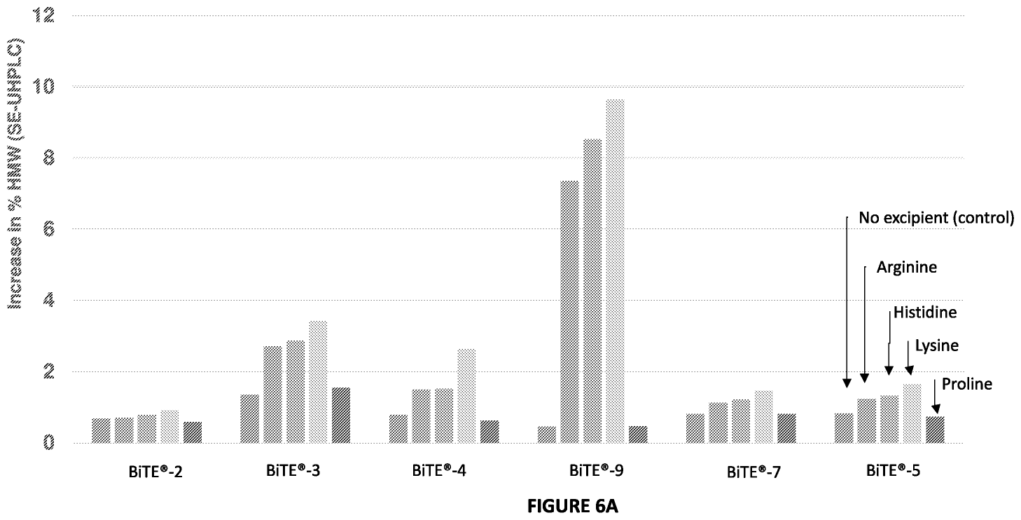

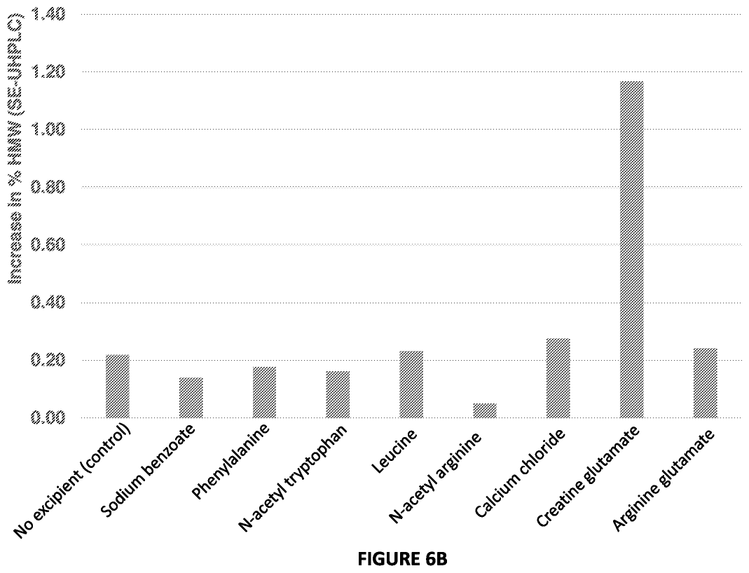

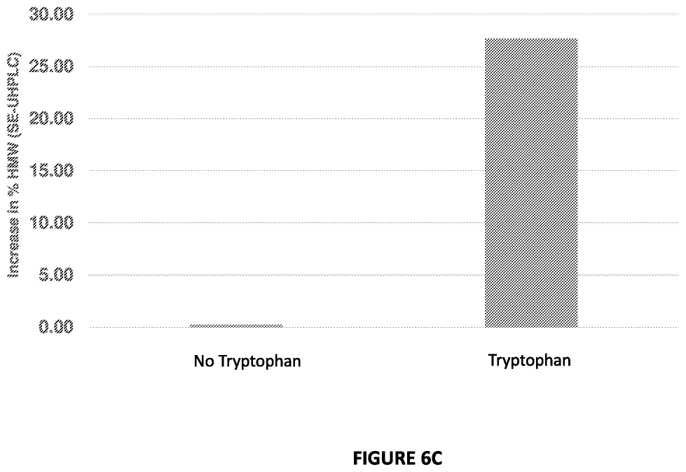

The addition of other amino acids and excipients to a formulation buffer comprising a BiTE molecule, 10 mM glutamate, 9% sucrose, 0.01% PS80 did not result in a significant decrease in frozen state aggregation levels after one-month storage at −20° C. (FIGS. 6A, 6B and 6C). All proteins were evaluated at 1 mg/mL. The amino acid concentration used in FIG. 6A was 10 mM and the excipient concentration used in FIGS. 6B and 6C was 50 mM.

The data provided in this Example demonstrate the stability of the formulation of the disclosure comprising methionine at −10° C. to −40° C. (e.g., −20° C. to −35° C., such as −30° C.) for a variety of bispecific antibody constructs. Interestingly, methionine did not significantly inhibit aggregation in liquid formulations or impact the stability of a lyophilized formulation.

### Example 2

Sample Preparation: An appropriate volume of 10 mM glutamate, 9% sucrose, 0.01% PS80, 200 mM methionine (pH 4.2) stock solution was added to 5 mg/mL BiTE®-5 (DLL3×CD3) (SEQ ID NO: 77) sample to achieve a final formulation of 10 mM glutamate, 9% sucrose, 0.01% PS80, pH 4.2 at varying methionine concentrations. The final protein concentration for BiTE®-5 was 2.5 mg/mL. All protein samples were staged at −20° C. for 24 hours to ensure complete freezing. The samples were then stored at −15° C. for 4 weeks. The t0 and stressed samples were evaluated for HMW content by SE-UHPLC.

SE-UHPLC Analysis: Stability samples were analyzed using SE-UHPLC (Size Exclusion Ultra High-Performance Liquid Chromatography) to monitor aggregation in the frozen state. Size Exclusion Ultra High-Performance Liquid Chromatography (SE-UHPLC) separates proteins based on differences in their hydrodynamic volumes. Molecules with higher hydrodynamic volumes elute earlier than molecules with smaller volumes. The samples are loaded onto an SE-UHPLC column (BEH200, 4.6×300 mm, (Waters Corporation, 186005226)), separated isocratically and the eluent is monitored by UV absorbance. Purity is determined by calculating the percentage of each separated component as compared to the total integrated area. SE-UHPLC settings are as follows: Flow rate: 0.4 mL/min, Run time: 12 min, UV detection: 280 nm, Column temperature: Ambient, Target protein load: 6 μg, Protein compatible flow cell: 5 mm.

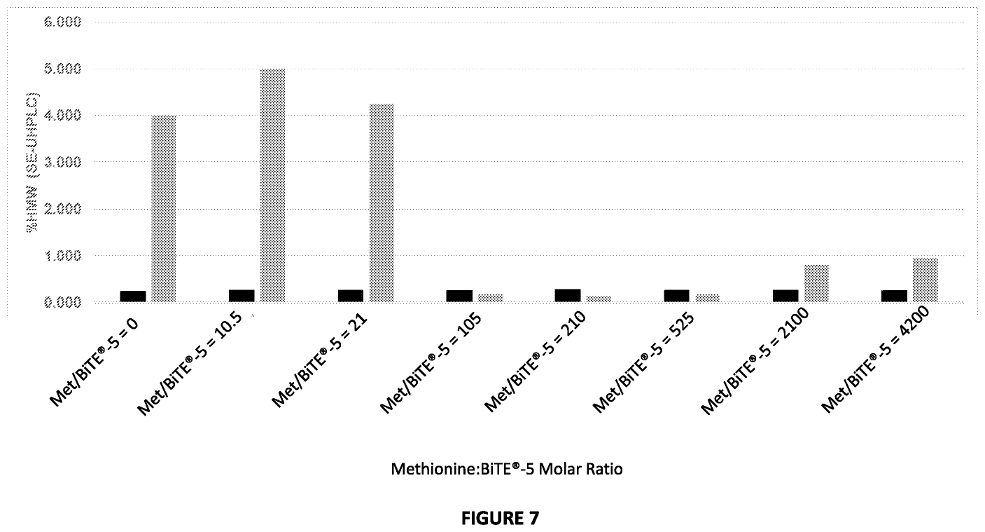

Results: A methionine to BiTE molar ratio of 105 and higher was observed to reduce frozen state aggregation of BiTE®-5 (FIG. 7). Ratios below 105 did not protect against frozen state aggregation to the extent observed using molar ratios of at least 105.

All of the references cited herein, including patents, patent applications, literature publications, and the like, are hereby incorporated in their entireties by reference.

While this invention has been described with an emphasis upon preferred embodiments, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

What is claimed is:

1. A pharmaceutical formulation comprising
(a) a bispecific antibody construct,
(b) a saccharide,
(c) a surfactant,
(d) a buffer, and
(e) methionine present at a molar ratio of methionine to bispecific antibody construct of about 10× to about 5000×;
wherein the pH of the formulation is from about 4 to about 7.

2. The formulation of claim 1, wherein the pH of the formulation is about 4.2.

3. The formulation of claim 1 or claim 2, wherein the saccharide is a monosaccharide or a disaccharide.

4. The formulation of any one of claims 1-3, wherein the saccharide is glucose, galactose, fructose, xylose, sucrose, lactose, maltose, trehalose, sorbitol, mannitol, or xylitol.

5. The formulation of claim 4, wherein the saccharide is sucrose.

6. The formulation of any one of claims 1-5, wherein the surfactant is a nonionic surfactant.

7. The formulation of any one of claims 1-6, wherein the surfactant is polysorbate 20, polysorbate 40, polysorbate 60, polysorbate 80, poloxamer 188, poloxamer 407, or Triton™ x-100.

8. The formulation of claim 7, wherein the surfactant is polysorbate 20, polysorbate 40, polysorbate 60, or polysorbate 80.

9. The formulation of claim 7, wherein the surfactant is polysorbate 80.

10. The formulation of any one of claims 1-9, wherein the buffer is an acetate buffer, a glutamate buffer, a citrate buffer, a lactic buffer, a succinate buffer, a tartrate buffer, a fumarate buffer, a maleate buffer, a histidine buffer, or a phosphate buffer.

11. The formulation of claim 10, wherein the buffer is a glutamate buffer.

12. The formulation of any one of claims 1-11, wherein the formulation comprises the bispecific antibody construct at a concentration of from about 1 mg/ml to about 20 mg/ml.

13. The formulation of any one of claims 1-12, wherein the formulation comprises methionine at molar ratio of methionine to bispecific antibody construct of about 50× to about 5000×.

14. The formulation of any one of claims 1-12, wherein the formulation comprises about 10 mM to about 200 mM methionine.

15. The formulation of any one of claims 1-14, wherein the formulation is frozen.

16. The formulation of any one of claims 1-14, wherein the formulation is a thawed formulation.

17. The formulation of any one of claims 1-14, wherein the formulation is lyophilized.

18. The formulation of any one of claims 1-17, which comprises at least about 10% less high molecule weight (HMW) species compared to a matched formulation not comprising methionine when stored for four weeks at −15° C.

19. The formulation of claim 18, which comprises at least about 25% to about 85% less high molecule weight (HMW) species compared to a matched formulation not comprising methionine when stored for four weeks at −15° C.

20. A frozen pharmaceutical formulation comprising about 1 mg/mL to about 20 mg/mL bispecific antibody construct, sucrose, glutamic acid, polysorbate 80, and about 10 mM to about 200 mM methionine, wherein the pH of the formulation is from about 4 to about 7.

21. The formulation of any one of claims 1-20, wherein the bispecific antibody construct comprises a first binding domain that binds to a target cell surface antigen and a second binding domain that binds to human CD3 on the surface of a T cell.

22. The formulation of claim 21, further comprising a third domain comprising, in an amino to carboxyl order, hinge-CH2 domain-CH3 domain-linker-hinge-CH2 domain-CH3 domain.

23. The formulation of claim 22, wherein each of the first and second binding domains comprise a VH region and a VL region.

24. The formulation of claim 22 or claim 23, wherein the bispecific antibody construct is a single chain antibody construct.

25. The formulation of any one of claims 21-24, wherein the target cell surface antigen is CDH19, MSLN, DLL3, FLT3, EGFR, EGFRvIII, BCMA, PSMA, CD33, CD19, CD70, MUC17, or CLDN18.2.

26. The formulation of any one of claims 21-25, wherein the bispecific antibody construct comprises the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

27. The formulation of any one of claims 1-26, wherein the formulation comprises methionine at molar ratio of methionine to bispecific antibody construct of about 105× to about 5000×, and the bispecific antibody construct comprises the amino acid sequence set forth in SEQ ID NO: 77.

28. A method of treating cancer in a subject in need thereof comprising administering the formulation of any one of claims 1-26 to the subject.

29. The method of claim 28, wherein the formulation is frozen and the method further comprises thawing the formulation prior to administration to the subject.

30. The method of claim 27 or 28, wherein the method comprises intravenously administering the formulation to the subject.

31. The method of any one of claims 28-30, wherein the formulation comprises methionine at molar ratio of methionine to bispecific antibody construct of about 105× to about 5000×, and the bispecific antibody construct comprises the amino acid sequence set forth in SEQ ID NO: 77.

32. A method comprising
(a) preparing a formulation comprising a bispecific antibody construct, methionine, and a buffer, wherein the methionine is present at a molar ratio of methionine to bispecific antibody construct of about 10× to about 5000×;
(b) freezing the formulation of (a); and
(c) storing the formulation of (b) at a temperature of about −10° C. to about −40° C.

30. The method of claim 29, wherein the method further comprises
(d) thawing the formulation of (c); and
(e) lyophilizing the formulation of (d).

33. The method of claim 32, wherein the formulation of (a) further comprises a saccharide, and comprises pH of from about 4 to about 7.

34. The method of claim 33, wherein between step (d) and (e) a buffer exchange step is performed to produce a pharmaceutical formulation comprising the bispecific antibody construct, a saccharide, a surfactant, a buffer, and methionine present at a molar ratio of methionine to bispecific antibody construct of about 10× to about 5000×; wherein the pH of the formulation is from about 4 to about 7.

35. The method of any one of claims 32-34, wherein steps (b) and (c) are performed at a temperature of about −20° C. to about −35° C.

36. The method of any one of claims 32-35, wherein step (c) comprises storing the formulation for at least one month.

37. The method of any one of claims 32-36, wherein the formulation comprises methionine present at molar ratio of methionine to bispecific antibody construct of about 50× to about 5000×

38. The method of any one of claims 32-37, wherein the formulation comprises about 10 mM to about 200 mM methionine.

39. The method of any one of claims 33-38, wherein the saccharide is a monosaccharide or a disaccharide.

40. The method of any one of claims 33-39, wherein the saccharide is glucose, galactose, fructose, xylose, sucrose, lactose, maltose, trehalose, sorbitol, mannitol, or xylitol.

41. The method of claim 40, wherein the saccharide is sucrose.

42. The method of any one of claims 34-41, wherein the surfactant is a nonionic surfactant.

43. The method of any one of claims 34-42, wherein the surfactant is polysorbate 20, polysorbate 40, polysorbate 60, polysorbate 80, poloxamer 188, poloxamer 407, or Triton™ x-100.

44. The method of claim 43, wherein the surfactant is polysorbate 80.

45. The method of any one of claims 32-44, wherein the buffer is an acetate buffer, a glutamate buffer, a citrate buffer, a lactic buffer, a succinate buffer, a tartrate buffer, a fumarate buffer, a maleate buffer, a histidine buffer, or a phosphate buffer.

46. The method of claim 45, wherein the buffer is a glutamate buffer.

47. The method of any one of claims 31-46, wherein the formulation comprises the bispecific antibody construct at a concentration of from about 1 mg/ml to about 20 mg/ml.

48. The method of any one of claims 31-47, wherein the bispecific antibody construct comprises a first binding domain that binds to a target cell surface antigen and a second binding domain that binds to human CD3 on the surface of a T cell.

49. The method of claim 48, further comprising a third domain comprising, in an amino to carboxyl order, hinge-CH2 domain-CH3 domain-linker-hinge-CH2 domain-CH3 domain.

50. The method of claim 49, wherein each of the first and second binding domains comprise a VH region and a VL region.

51. The method of claim 49 or claim 50, wherein the bispecific antibody construct is a single chain antibody construct.

52. The method of any one of claims 48-51, wherein the target cell surface antigen is CDH19, MSLN, DLL3, FLT3, EGFR, EGFRvIII, BCMA, PSMA, CD33, CD19, CD70, MUC17, or CLDN18.2.

53. The method of any one of claims 48-52, wherein the bispecific antibody construct comprises the amino acid sequence set forth in SEQ ID NO: 20, SEQ ID NO: 21, SEQ ID NO: 22, SEQ ID NO: 23, SEQ ID NO: 33, SEQ ID NO: 43, SEQ ID NO: 44, SEQ ID NO: 45, SEQ ID NO: 55, SEQ ID NO: 65, SEQ ID NO: 66, SEQ ID NO: 76, SEQ ID NO: 77, SEQ ID NO: 87, SEQ ID NO: 97, SEQ ID NO: 98, SEQ ID NO: 99, SEQ ID NO: 109, SEQ ID NO: 110, SEQ ID NO: 111, SEQ ID NO: 121, SEQ ID NO: 122, SEQ ID NO: 131, SEQ ID NO: 141, SEQ ID NO: 142, SEQ ID NO: 146, SEQ ID NO: 147, SEQ ID NO: 156, SEQ ID NO: 165, SEQ ID NO: 174, SEQ ID NO: 183, SEQ ID NO: 184, SEQ ID NO: 185, SEQ ID NO: 186, SEQ ID NO: 187, or SEQ ID NO: 188.

54. The method of claim 53, wherein the formulation comprises methionine at molar ratio of methionine to bispecific antibody construct of about 105× to about 5000×, and the bispecific antibody construct comprises the amino acid sequence set forth in SEQ ID NO: 77.
